(19) United States
(12) Patent Application Publication
(10) Pub. No.: US 2024/0355475 A1
(43) Pub. Date: Oct. 24, 2024

(54) GUM DISEASE EXAMINATION

(71) Applicant: KONINKLIJKE PHILIPS N.V., EINDHOVEN (NL)

(72) Inventors: Gerben KOOIJMAN, Leende (NL); Dionisio Massingo NUNES, Utrecht (NL); Amir Hussein RMAILE, Eindhoven (NL)

(21) Appl. No.: 18/684,670

(22) PCT Filed: Aug. 10, 2022
(86) PCT No.: PCT/EP2022/072409

§ 371 Date: Feb. 17, 2024

Related U.S. Application Data
(60) Provisional application No. 63/233,937, filed on Aug. 17, 2021.

(51) Int. Cl. G16H 50/20; G16H 10/60; G16H 50/70

(52) U.S. Cl. CPC G16H 50/20; G16H 10/60; G16H 50/70

(57) ABSTRACT

A system for assisting a gum disease examination provides indication of an oral area to be examined during an examination of the gums of a subject under diagnosis. Historical examination data is used in respect of a set of subjects. Using this data, a first oral area to be examined is determined, and based on the historical examination data and the measurement for any previously examined oral areas during the examination, a next oral area to be examined is also determined. In this way, a sequence of oral areas to be examined is determined by the system, with the aim of making the examination as quick as possible, by obtaining a diagnosis after as few measurements as possible (when there is a positive diagnosis to be made).

### FIELD OF THE INVENTION

This invention relates to a system for assisting a gum disease examination, such as a periodontitis examination.

### BACKGROUND OF THE INVENTION

Periodontitis is a chronic gum disease which has been reported as affecting approximately half of the adult US population, yet more than half of those affected are not diagnosed as such by their dentist. The key reason is that the screening process is time consuming and painful and can only be done by a dental practitioner.

A dental practitioner will typically perform a so-called “Full-Mouth Periodontal Examination” (FMPE) to determine the correct intervention needed by a patient. The procedure involves probing four or six sites per tooth on 28 teeth (wisdom teeth are excluded), amounting to 112 or 168 sites being probed.

During a periodontal examination, each tooth is assessed individually. For probing with a graduated periodontal probe (e.g. with markings at 3, 6, 8, and 11 mm), a slight force of 0.25 N (25 g) should be applied. The tip of the probe (diameter 0.5 mm) is inserted gently along the axis of the tooth into the gingival sulcus. The probing depth is read from the markings on the probe.

FMPE is only relevant for patients known to have the disease. To determine if a patient is a candidate for FMPE, a dental practitioner will often perform a Partial-Mouth Examination, in which a selected number of sites are probed. In 1993, the American Dental Association (ADA) introduced the Periodontal Screening and Recording (PSR) system, as a quick, reproducible method for identifying patients that require FMPE. The PSR uses numeric codes to represent gum disease: gingivitis codes 0 to 2; periodontitis codes 3 to 4.

In the PSR system, a special probe (“PSR Probe”) is used to probe each site in a sextant, but only the largest pocket depth measured in the sextant is recorded. Each measured value is associated with one of the numeric codes, using a specially defined threshold. The main advantage of the PSR over FMPE is that a simpler probe is used and the dental practitioner only needs to record six numbers.

Typically, a dental practitioner will measure a few sites in a sextant and will stop when they feel the measurements are within normal limits. This practice is very common and often fails to accurately assess the patients' health.

It is worth noting that mild and moderate periodontitis account for 81% of all periodontal cases, yet they are most often not diagnosed as such. The fact that severe patients are diagnosed at a higher rate shows that the likelihood of diagnosis increases when it is already too late.

There is a need for a reliable way to diagnose gum disease as efficiently as possible, in particular to reduce the discomfort to a patient of a prolonged examination.

### SUMMARY OF THE INVENTION

The invention is defined by the claims.

According to examples in accordance with an aspect of the invention, there is provided a system for assisting a gum disease examination, comprising:
an output for indicating an oral area to be examined during an examination of the gums of a subject under diagnosis;
a first input for receiving historical examination data in respect of a set of subjects, the historical examination data identifying oral areas that have been examined and the associated examination measurements;
a second input for receiving examination results of an indicated oral area; and
a processor, wherein the processor is adapted to:
   determine a first oral area to be examined based on the historical examination data;
   determine whether the examination of an indicated oral area or areas during the examination so far meets measurement criteria indicative of gum disease; and
   determine, based on the historical examination data and the examination of an indicated oral area or areas during the examination so far, a next oral area to be examined.

The invention provides a system and software-implemented method to ensure the quickest path to establish a diagnosis, by dynamically making use of a database of historical examination data for gum health condition. The database typically contains detailed information for a section of the mouth, individual teeth or even individual tooth sites. Thus, each oral area may comprise a section of the mouth, an individual tooth, or an individual tooth site.

The system is applicable to a professional dental practice as well to a consumer for self-diagnosis.

In a dental practitioner practice, real-time guidance is given in the disease screening process, significantly reducing the time required compared to the current standard. In a consumer application, the method can be used to optimally guide the patient in using a diagnosis tool (for example a tool based on taking intra-oral images) for periodontal self-screening.

The system minimizes the time and effort involved in periodontal examination. Real-time guidance is given for example by showing which oral area the user of the system needs to assess next, in order to come to a speedy diagnosis. This is for example done dynamically by means of a probabilistic algorithm that recommends, in real-time, examination areas based on measurements taken from a previous site.

The use of measurements for previously examined oral areas of the currently performed examination, in order to decide the next oral area for examination, provides a more personalized sequence. For example, gum disease (e.g. periodontitis) is typically somewhat localized in certain areas in the mouth. Over the whole population pocket depth and bleeding is usually worse on the far molar sites and lower front inner sites. Nevertheless, this localization can differ between individuals. Thus, when examining a patient, the method basically tries to find similar patients in the historical database, from which the most likely worst condition sites are determined.

The system may further comprise a third input for receiving patient metadata, and wherein the processor takes account of the patient metadata in determining the oral areas to be examined. This allows certain factors, such as smoking, age, etc. to be taken into account. The historical examination data preferably also includes patient metadata so that a subject being diagnosed can be matched better to the historical data.

The examination may be periodontal probing (by a dentist), X-ray imaging (by a dentist) or visible light imaging (by a consumer).

The processor may be adapted to indicate an end of the examination when:
the examination of the so far indicated and measured oral areas meets measurement criteria indicative of gum disease; or
sufficient oral areas have been examined such that the measurement criteria indicative of gum disease can no longer be met.

Thus, the examination ends as soon as a positive diagnosis is given, or when a sufficient oral areas have been examined that a positive diagnosis is no longer possible, i.e. the criteria for gum disease can no longer be met given the measurement results of examined oral areas so far and any possible measurement results on not examined oral areas. Thus, the examination time is kept to a minimum.

The processor may be adapted to determine a first oral area to be examined and any next oral areas to be examined based on statistical analysis of the historical examination data. For example, the statistical analysis may identify a probability of each examination oral area being included in a positive gum disease diagnosis. This increases the chances of a short examination (when there is gum disease identified).

The system may further comprise a display for providing real-time guidance illustrating the oral area to be examined.

The system may comprise a database storing the historical examination data. Alternatively, the system may access a remote database.

A fourth input may be provided for receiving intra-oral scan data for the patient, and wherein the processor takes account of the patient intra-oral scan data in determining the oral areas to be examined.

In this way, comparison of the current patient's scan data with scan data of patients in the database is used in determining the next recommended oral area to be examined (e.g. by probing).

The invention also provides a computer program comprising computer program code means which is adapted, when said program is run on a computer, to implement a method comprising:
using historical examination data in respect of a set of subjects, the historical examination data identifying oral areas that have been examined and the associated examination measurements, to determine a first oral area to be examined;
providing as an output an indication of the first oral area to be examined;
receiving examination results of an indicated oral area;
determining whether the examination of an indicated oral area or areas during the examination so far meets measurement criteria indicative of gum disease; and
if the examination of the so far indicated oral areas does not meet the measurement criteria, determining, based on the historical examination data and the examination of an indicated oral area or areas during the examination so far, a next oral area to be examined.

The program thus guides a user to examine the oral areas in an order that is most likely to result in a short examination process.

The method may comprise indicating an end of the examination when:
the examination of the so far indicated and measured oral area meets measurement criteria indicative of gum disease; or
sufficient oral areas have been examined such that the measurement criteria indicative of gum disease can no longer be met.

The determining of a first oral area and any next oral areas to be examined is for example based on statistical analysis of the historical examination data.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows an example of a set of six probing sites for a single tooth;

FIG. 2 shows that for a full mouth periodontal examination (FMPE) all teeth (excluding wisdom teeth) are probed and a full set of probe data is obtained.

FIG. 3 shows that for Periodontal Screening and Recording (PSR), six sextants are defined;

FIG. 4 shows the likelihood of periodontitis being diagnosed by a dental practitioner, in dependence on the level of severity;

FIG. 5 shows a method for assisting a gum disease examination;

FIG. 6 shows an example of a possible image used as part of a user interface;

FIG. 7 shows an example of the statistical data which can be derived from the historical examination data;

FIG. 8 shows a system for assisting a gum disease examination; and

FIG. 9 illustrates an example of a computer which may be used to perform the function of the processor shown in FIG. 7.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides a system for assisting a gum disease examination which provides indication of an oral area to be examined during an examination of the gums of a subject under diagnosis. Historical examination data is used in respect of a set of subjects. Using this data, a first oral area to be examined is determined, and based on the historical examination data and also the measurement for any previously examined oral areas during the examination, a next oral area to be examined is also determined. In this way, a sequence of oral areas to be examined is determined by the system, with the aim of making the examination as quick as possible, by obtaining a diagnosis after as few measurements as possible (when there is a positive diagnosis to be made).

The invention may be applied to a periodontal examination using a probe, or it may be applied to examination based on imaging.

The invention relates generally to the assessment of gum disease, of which periodontitis and gingivitis are examples. Both periodontitis and gingivitis can typically be assessed with probing. The invention is of particular interest for periodontitis assessment.

For the degree of periodontitis (i.e. healthy, mild periodontitis, moderate periodontitis, severe periodontitis), criteria on probing pocket depth and attachment loss are usually applied. Some definitions also include criteria on bleeding-on-probing (i.e. whether or not the site bleeds when probed). For gingivitis, typically criteria are used related to bleeding-on-probing only, i.e. pocket depth and/or attachment loss are not relevant for gingivitis diagnosis.

An example for periodontitis definition based on pocket depth (PC) and Clinical Attachment Loss (CAL) is that used by the American Academy of Periodontology (AAP) and Centers for Disease Control and Prevention (CDC):

### Severe Periodontitis:

(at least 1 site with PC>=5) AND (at least 2 teeth with CAL>=6 at at least one site)

### Moderate Periodontitis:

[NOT severe] AND
[(at least 2 teeth with at least 1 site with PC>=5) OR
   (at least 2 teeth with at least 1 site with CAL>=4)]

### Mild Periodontitis:

[NOT severe AND NOT moderate] AND
[{1 site with PC>=5} OR
{(at least 2 teeth with at least one site with PC>=4) AND (at least 2 sites with CAL>=3)}]

### Healthy

### NOT Severe AND NOT Moderate AND NOT Mild

Other ways of interpreting probing depths, bleeding and other factors in order to produce a scale of severity are also of course possible.

An application of the invention to the use of periodontal probe measurements will first be described.

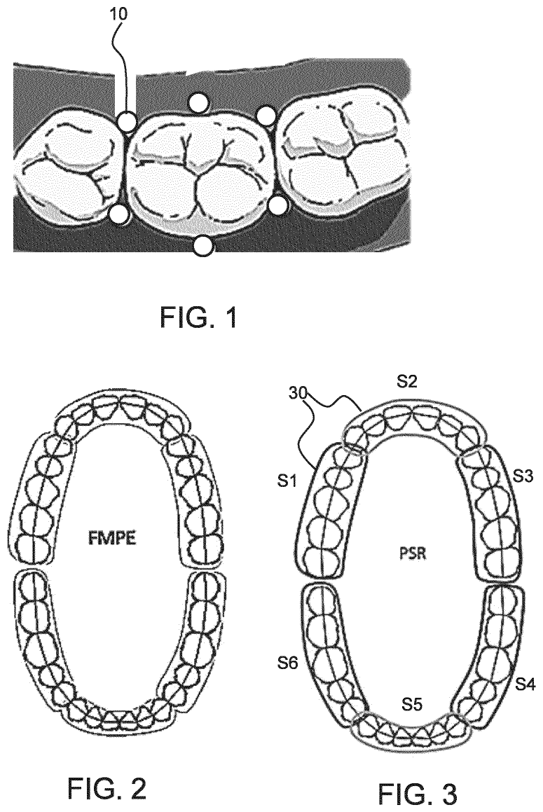

FIG. 1 shows an example of a set of six probing sites 10 for a single tooth. Typically, a periodontal examination involves making probe measurements at all six sites for each tooth in turn.

FIG. 2 shows that for a full mouth periodontal examination (FMPE) all teeth (excluding wisdom teeth) are probed and a full set of probe data is obtained. This for example involves 168 probe measurements.

FIG. 3 shows that for Periodontal Screening and Recording (PSR), six sextants 30 are defined S1 to S6 (some with 5 teeth and some with 6 teeth) and only the deepest site in each sextant is recorded.

For PSR all sites are still probed, i.e. 6 sites per tooth on all teeth. The difference with FMPE is the extent of recording the measurements (and consequently the gum disease criteria used): for PSR only the worst site for each sextant is recorded, whereas for FMPE all sites are recorded. Hence, the lower effort with PSR is due to less recording.

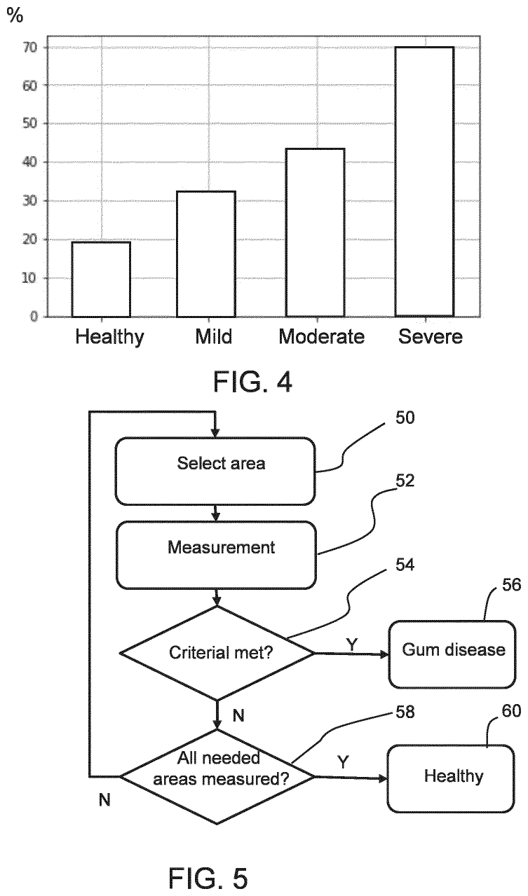

FIG. 4 shows the general likelihood of periodontitis being diagnosed by a dental practitioner, in dependence on the level of severity.

The y-axis shows the percentage chance of a correct positive diagnosis for the different levels of severity along the x-axis. It shows that a reliable diagnosis is typically only made when the condition has already become severe. There is therefore a need for a more reliable diagnostic tool, which enables the discomfort to the user to be kept to a minimum.

FIG. 5 shows a method for assisting a gum disease examination in accordance with an embodiment of the invention.

In step 50, an initial oral area to be examined is determined by software running on a processor of a system for assisting the gum disease examination. This initial area is output by the system as a recommendation for a starting oral area for examination. For a periodontal probing system, this indicates a tooth site to be probed. However, this oral area could be a whole tooth, or even a larger general area of the mouth.

The recommendation is essentially based on how likely it is that the particular area will lead to a diagnosis, given a particular diagnosis criteria.

A selection algorithm running on a processor is used for this purpose, which takes as input a sufficiently representative data set, containing probing data and metadata of a set of subjects (age, gender, medical conditions, influencing factors etc.), the corresponding metadata of the subject under diagnosis, and the chosen gum disease diagnosis criteria. In this way, historical examination data in respect of a set of subjects is received by the selection algorithm. The historical examination data identifies oral areas that have historically been examined and the associated examination measurements, e.g. probe depth measurements for the example of a periodontal probe.

A measurement is then done in step 52 by the user of the system (e.g. a dental practitioner) at the selected recommended site. The measurement results are provided to the processor.

In step 54 it is determined if the measurements so far (i.e. measured probe data for the latest measurement and previous measurements) meets the gum disease criteria. If the criterial is met, there is a positive diagnosis of the gum disease being tested, in step 56. If the gum disease criteria are not met, it is checked in step 58 whether sufficient sites have been measured to rule out a positive diagnosis. This will typically not require all sites to have been measured. For example, this applies when the criteria for gum disease can no longer be met given the measurement results of the examined oral areas so far and any possible measurement results on not-examined oral areas. Thus, the examination time is kept to a minimum. When sufficient sites have been measured, the diagnosis is healthy in step 60, with no detected gum disease (of the type specified).

The number of sites tested will thus depend on the criteria used and the measurement results themselves. In practice, it is desired to determine a positive outcome, e.g. for a certain degree of periodontitis, as quickly as possible. For example, if looking for any form of periodontitis versus healthy, the criteria discussed above may be used. In this case a measurement at only one site may be needed, namely, a site with PC>=5 already indicates (at least) mild periodontitis. In the same way, all sites may need to be probed to come to the conclusion that none fulfill this condition, and the diagnosis is healthy.

If a positive diagnosis cannot yet be ruled out, the method returns to step 50 and a next site is selected, again using the selection algorithm. This algorithm now takes as input the historical data set and the metadata of the subject under diagnosis (as before) but also the measured probing data of the subject under diagnosis that has already been completed.

By using the measured probing data of the current examination in order to make a selection of the next probing site, the sequence is personalized with the aim of identifying the most likely worst condition sites and assessing them in order, with the most likely sites for a positive diagnosis first. A currently measured pattern of measured probing data can be compared with the historical data to identify similar patients in the database and more accurately identify the next most likely site for a positive diagnosis. The algorithm thus bases its next recommendation on the preceeding measurements so that every new measurement can alter the sequence the algorithm recommends.

When a definite diagnosis is reached, the patient's probing data and metadata may be added to the database of examination data, so that the reference database will increase in size over time. By updating the historical database in real-time, the system benefits from data taken from individuals who might not be well represented in the base dataset used to create the algorithm.

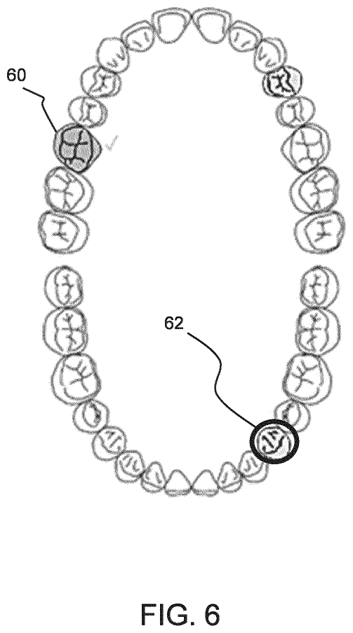

FIG. 6 shows an example of a possible image used as part of a user interface. It shows all the teeth. The already-examined teeth 60 are shown in one form, such as with a color, and a next-to-be-examined tooth 62 is shown in another form, such as surrounded by a circle. Individual teeth may be identified (as shown) or individual tooth sites may be identified, or else a more general oral area (for imaging) may be shown.

The system thus uses a display to provide real-time guidance illustrating the oral area to be examined.

The algorithm used for recommending the oral area to be examined (i.e. the first area, and then any subsequent areas while a positive diagnosis has not been made) can be based on known statistical methods. These may for example comprise (naïve) Bayesian classification, which considers conditional probabilities.

The simplest form of algorithm may be a function that scores each tooth site according to how often that site has been included in all positive diagnoses of the gum disease being diagnosed e.g. periodontitis. The highest scoring tooth site can then be selected as the starting site.

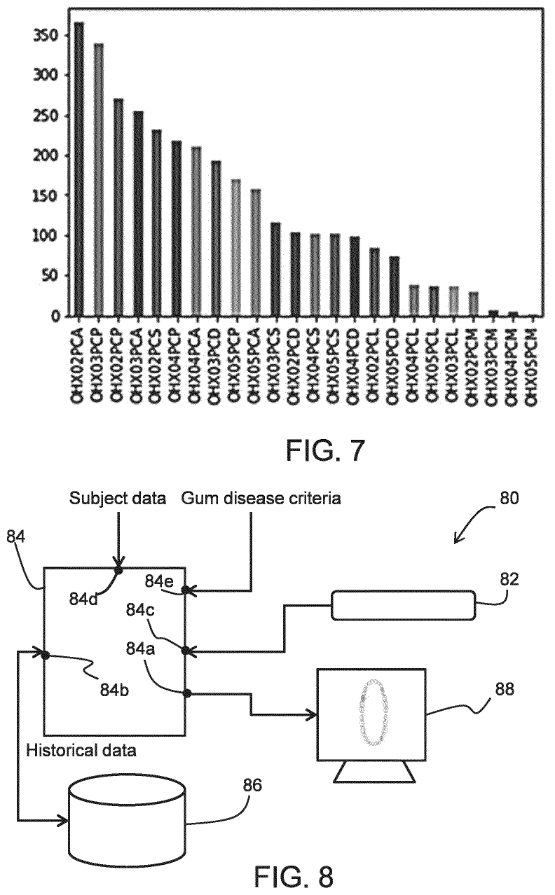

FIG. 7 shows an example of the statistical data which can be derived from the historical examination data.

The x-axis shows codes for the 24 tooth sites present in sextant 1. Sextant 1 has 5 teeth (for a subject with no missing teeth), but the third molar is not investigated, giving 4×6=24 measurements. Numbers 02 to 05 represent the upper left molars excluding the third molar.

The y-axis shows a ranking of how many times each tooth site was included in a positive periodontitis diagnosis.

In addition, weights can be added to the probabilities allocated to each site. These may be derived from parameters or metadata that are known to influence the pocket depth at a particular site, such as smoking.

A dental practitioner may for example be recommended to probe sites in the front sextants mostly for patients who smoke as a result of this weighting function, as deeper pockets are rare in the front sextant for non-smoking patients (of the selected age group). In principle, any data about the patient known to impact pocket depth can be used in the calculation of weights used in the ranking of the sites.

Another algorithm may search for sites having the highest pocket depth within a sextant, based on the historical data. This is especially relevant for the PSR method described above, which is based on pocket depth measurement only, and which looks only to the largest pocket depth per sextant.

The selection of the first site to measure as well as subsequent sites to measure is based on the historical data. Within that data, measurement results for subjects in the set being more similar to the individual being diagnosed are also weighted more heavily. Thus, the patient metadata is used as part of the statistical analysis.

By way of example, an algorithm for the next site selection will now be outlined in more detail, based on a similarity between the subject under diagnosis and patients in the historical database:
(i) For selection of a next site, first, a comparison between subjects in the historical database and the subject being diagnosed is made with respect to the measured probing data and (optionally) metadata (e.g. gender, age, smoking). For selection of the starting site, no probing data of the subject being diagnosed is measured yet. In that case, comparison based on only metadata can be done.

For this comparison, a distance metric Di is calculated for each subject i in the dataset based on the similarity/difference of considered (meta) data between that subject and the subject being diagnosed.

When there is a number of K features with values fₛ^{k} for the diagnosed individual and fᵢ^{k} for the subjects in the dataset (with k=1 . . . . K), the distance metric may for instance be calculated as the L2 norm:

$${D}_{i} = {\sum }_{k = 1}^{K} {( {f}_{i}^{k} - {f}_{s}^{k} )}^{2} .$$

Alternative formulae may be used where the range of values per feature is taken into account and/or specific features weigh more strongly in calculating the distance, e.g.:

$${D}_{i} = {\sum }_{k = 1}^{K} {{w}^{k} ( {f}_{i}^{k} - {f}_{s}^{k} )}^{2}$$

with w^{k} a pre-determined weighting factor per feature [symbol] .

For categorical features (e.g. yes/no smoking) conversion to a numerical feature value [symbol] is done (e.g. 0=not smoking, 1=smoking).

Note that if no metadata is taken into account in the method, and the starting site is being selected (with no probing data of the diagnosed subject available yet) there are no features yet to compare: in that case the distance metric is set equal for all subjects in the data set (e.g. at a value of 0).
(ii) Next for each site j, which is not yet measured for the subject being diagnosed, a score is determined. This may for instance be done by the following equation:

$$score ( {s}_{j} ) = {\sum }_{i = 1}^{N} W ( {D}_{i} ) \{ \begin{matrix}1 if PD ( {s}_{j} ) = {PD}_{max , i} \\ 0 otherwise\end{matrix}$$

This means that for each individual i in the historical database, each site sⱼ (not yet measured for the subject being diagnosed) is investigated, and it is checked if, for that site, the pocket depth PD equals the maximum pocket depth PD_{max,i} for that subject. If so, a value of 1 is assigned to that site.

This is performed for all N subjects in the historical database, such that the scores over all individuals are summed. However, in doing so, a weight W(Dᵢ) may be taken into account. This weight varies per subject i as it depends on the previously determined distance metric Dᵢ. The function W(Dᵢ) is in general chosen such that a smaller distance Dᵢ will give a larger weight W. In this way, subjects in the historical database that are more similar to the subject being diagnosed weigh more strongly in the next site selection.
(iii) The next site to be measured is then simply determined as the site having the highest score:

$${s}_{m} = {arg}_{j} max ( score ( {s}_{j} ) )$$

(iv) If the measured site does not lead to a diagnosis yet, a next site to be measured is again determined, starting again at step (i). The distance metric is re-calculated as now an additional feature is available, i.e. the pocket depth of the just measured site. After that at step (ii), the scorings are re-calculated for the current remaining unmeasured sites, taking into account the re-calculated distance metric. The next site is then selected at step (iii) as the site with highest score.

Note that when using the PSR criterium, sites in a sextant may not need to be measured further once a site with certain pocket depth is already found in that sextant. The algorithm may be adapted that for such cases the next site is selectable only from the sextants not yet completed.

FIG. 8 shows a system 80 for assisting a gum disease examination. The system comprises an examination device 82 such as a periodontal probe (or an imaging device as explained below) and a processor 84.

The processor has a first input 84b for receiving historical examination data in respect of a set of subjects (“historical data”) which identifies oral areas that have been examined and the associated examination measurements. This data comes from a historical data database 86. The historical data database can also contain metadata for the set of subjects.

A second input 84c receives the measurement from the examination device, so that the processor can determine if the measurements so far mean the criteria for a positive diagnosis have been met.

Subject metadata (“subject data”) is also provided as third input 84d, such as age, gender, and any relevant medical or other conditions or information of relevance. A fourth input 84e indicates criteria to be applied in making the gum disease diagnosis (“gum disease criteria”).

An output 84a is used to drive a display device 88. The output indicates an oral area to be examined during an examination of the teeth of the subject under diagnosis;

The processor 84 determines a first and subsequent oral areas to be examined as explained above.

Additional periodontal related data may be included in the screening method. For example, intra-oral scanner (IOS) data may be obtained for the patient before the probing procedure is started. This uses visible light imaging to create a 3D map of the teeth, for example using structured light. Comparison of the IOS data for the current patient with IOS data of patients in the historical database may then additionally be used by the algorithm for selecting the areas to be examined.

The example above is based on the use of a periodontal probe. However, the site recommendation method may instead be used for X-ray imaging instead of periodontal pocket probing.

The first recommended oral area to be examined then is used is for recording an X-ray image, which is then analyzed by image analysis to test the desired diagnosis. Again, the recommended site has the highest probability to provide a diagnosis. If no periodontal diagnosis can be made based on this recorded X-ray, a next location is recommended based on comparison of the metadata and the previously recorded X-ray with the database. The oral area to be examined may then be an individual tooth or a larger area, rather than a tooth site as in the case of a probe.

Another implementation may be based on self-screening by a consumer. For example, a consumer-operable device may take intra-oral images (using visible light) at certain locations in the mouth, and a periodontal diagnosis may be based on analyzing the images. The invention may again be used to provide a recommendation of the first in-mouth location for taking an image. The recommended location again has the highest probability of giving a definite diagnosis.

In addition to age, gender, and smoking habits as mentioned above, the metadata may include brushing data obtained via a sensor-enabled toothbrush system. This brushing data for example includes coverage of the different mouth segments as well as pressure and scrubbing behavior at the different mouth segments.

If the image recorded at the first recommended site does not provide a diagnosis, a next site most likely to provide a diagnosis is recommended. This is based on the same metadata and brushing data as well as the previously recorded image.

As in the examples above, after completing the procedure the obtained data from the consumer or patient may be added to the historical database.

The invention is thus of interest for professional as well as consumer products and services for periodontal disease screening.

As discussed above, the system makes use of processor to perform the data processing. The processor can be implemented in numerous ways, with software and/or hardware, to perform the various functions required. The processor typically employs one or more microprocessors that may be programmed using software (e.g., microcode) to perform the required functions. The processor may be implemented as a combination of dedicated hardware to perform some functions and one or more programmed microprocessors and associated circuitry to perform other functions.

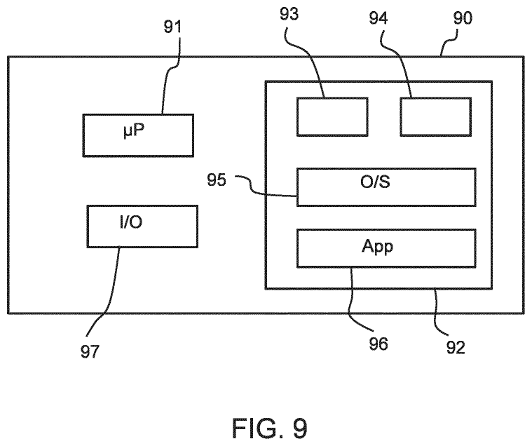

By way of further example, FIG. 9 illustrates an example of a computer 90 which may be used to perform the function of the processor shown above. It to be understood that system functional blocks can run on a single computer or may be distributed over several computers and locations (e.g. connected via internet).

The computer 90 includes, but is not limited to, PCs, workstations, laptops, PDAs, palm devices, servers, storages, and the like. Generally, in terms of hardware architecture, the computer 90 may include one or more processors 91, memory 92, and one or more I/O devices 97 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 91 is a hardware device for executing software that can be stored in the memory 92. The processor 91 can be virtually any custom made or commercially available processor, a central processing unit (CPU), a digital signal processor (DSP), or an auxiliary processor among several processors associated with the computer 90, and the processor 91 may be a semiconductor based microprocessor (in the form of a microchip) or a microprocessor.

The memory 92 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 92 may incorporate electronic, magnetic, optical, and/or other types of storage media. Note that the memory 92 can have a distributed architecture, where various components are situated remote from one another, but can be accessed by the processor 91.

The software in the memory 92 may include one or more separate programs, each of which comprises an ordered listing of executable instructions for implementing logical functions. The software in the memory 92 includes a suitable operating system (O/S) 95, compiler 94, source code 93, and one or more applications 96 in accordance with exemplary embodiments. As illustrated, the application 96 comprises numerous functional components for implementing the features and operations of the exemplary embodiments. The application 96 of the computer 90 may represent various applications, computational units, logic, functional units, processes, operations, virtual entities, and/or modules in accordance with exemplary embodiments, but the application 96 is not meant to be a limitation.

The operating system 95 controls the execution of other computer programs, and provides scheduling, input-output control, file and data management, memory management, and communication control and related services. It is contemplated by the inventors that the application 96 for implementing exemplary embodiments may be applicable on all commercially available operating systems.

Application 96 may be a source program, executable program (object code), script, or any other entity comprising a set of instructions to be performed. When a source program, then the program is usually translated via a compiler (such as the compiler 94), assembler, interpreter, or the like, which may or may not be included within the memory 92, so as to operate properly in connection with the O/S 95. Furthermore, the application 96 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 97 may include input devices such as, for example but not limited to, a mouse, keyboard, scanner, microphone, camera, etc. Furthermore, the I/O devices 97 may also include output devices, for example but not limited to a printer, display, etc. Finally, the I/O devices 97 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 97 also include components for communicating over various networks, such as the Internet or intranet.

If the computer 90 is a PC, workstation, intelligent device or the like, the software in the memory 92 may further include a basic input output system (BIOS) (omitted for simplicity). The BIOS is a set of essential software routines that initialize and test hardware at startup, start the O/S 95, and support the transfer of data among the hardware devices. The BIOS is stored in some type of read-only-memory, such as ROM, PROM, EPROM, EEPROM or the like, so that the BIOS can be executed when the computer 90 is activated.

When the computer 90 is in operation, the processor 91 is configured to execute software stored within the memory 92, to communicate data to and from the memory 92, and to generally control operations of the computer 90 pursuant to the software. The application 96 and the O/S 95 are read, in whole or in part, by the processor 91, perhaps buffered within the processor 91, and then executed.

When the application 96 is implemented in software it should be noted that the application 96 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 96 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems. (optional)

If the term “adapted to” is used in the claims or description, it is noted the term “adapted to” is intended to be equivalent to the term “configured to”.

Any reference signs in the claims should not be construed as limiting the scope.

1. A system for assisting a gum disease examination, comprising:
an output for indicating an oral area to be examined during an examination of the gums of a subject under diagnosis;
a first input for receiving historical examination data in respect of a set of subjects, the historical examination data identifying oral areas that have been examined and the associated examination measurements;
a second input for receiving examination results of an indicated oral area; and
a processor, wherein the processor is adapted to:
determine a first oral area to be examined based on the historical examination data;
determine whether the examination of an indicated oral area or areas during the examination so far meets measurement criteria indicative of gum disease; and
determine, based on the historical examination data and the examination of an indicated oral area or areas during the examination so far, a next oral area to be examined.

2. The system of claim 1, further comprising a third input for receiving patient metadata, and wherein the processor takes account of the patient metadata in determining the oral areas to be examined.

3. The system of claim 1, wherein the historical examination data further comprises patient metadata.

4. The system of claim 1, wherein the examination is periodontal probing.

5. The system of claim 1, wherein the examination is an imaging procedure.

6. The system of claim 1, wherein the processor is adapted to indicate an end of the examination when:
the examination of the so far indicated and measured oral areas meets measurement criteria indicative of gum disease; or
sufficient oral areas have been examined such that the measurement criteria indicative of gum disease can no longer be met.

7. The system of claim 1, wherein the processor is adapted to determine a first oral area and any next oral areas to be examined based on statistical analysis of the historical examination data.

8. The system of claim 7, wherein the statistical analysis identifies a probability of each examination oral area being included in a positive gum disease diagnosis.

9. The system of claim 1, further comprising a display for providing real-time guidance illustrating the oral area to be examined.

10. The system of claim 1, further comprising a database storing the historical examination data.

11. The system of claim 1, further comprising a fourth input for receiving intra-oral scan data for the patient, and wherein the processor takes account of the patient intra-oral scan data in determining the oral areas to be examined.

12. The system of any claim 1, wherein each oral area comprises a section of the mouth, an individual tooth, or an individual tooth site.

13. A computer program comprising computer program code means which is adapted, when said program is run on a computer, to implement a method comprising:
using historical examination data in respect of a set of subjects, the historical examination data identifying oral areas that have been examined and the associated examination measurements, to determine a first oral area to be examined;
providing as an output an indication of the first oral area to be examined;
receiving examination results of an indicated oral area;
determining whether the examination of an indicated oral area or areas during the examination so far meets measurement criteria indicative of gum disease; and
if the examination of the so far indicated oral areas does not meet the measurement criteria, determining, based on the historical examination data and the examination of an indicated oral area or areas during the examination so far, a next oral area to be examined.

14. The computer program of claim 13, wherein the method comprises indicating an end of the examination when:
the examination of the so far indicated and measured oral area meets measurement criteria indicative of gum disease; or
sufficient oral areas have been examined such that the measurement criteria indicative of gum disease can no longer be met.

15. The computer program of claim 14, wherein the method comprises determining a first oral area and any next oral areas to be examined based on statistical analysis of the historical examination data.
